(19) United States
(12) Patent Application Publication
(10) Pub. No.: US 2017/0283808 A1
(43) Pub. Date: Oct. 5, 2017

(54) MIR-520B SEQUENCE FOR INHIBITING CANCER GROWTH AND SPREAD AND MEDICINE THEREFOR

(71) Applicant: CHANG GUNG UNIVERSITY, TAOYUAN CITY (TW)

(72) Inventors: Ann-Joy Cheng, Taoyuan City (TW); Ya-Ching Lu, New Taipei City (TW)

(21) Appl. No.: 15/085,528

(22) Filed: Mar. 30, 2016

(51) Int. Cl. C12N 15/113; A61K 9/00; A61K 31/7105

(52) U.S. Cl. CPC C12N 15/1138; A61K 31/7105; A61K 9/0019; C12N 2320/30; C12N 2310/141

(57) ABSTRACT

A miR-520b sequence for inhibiting cancer growth and spread includes miR-520b sequence for inhibiting cancer growth and spread wherein the miR-520b sequence is 5′-AAAGUGCUUCCUUUUAGAGGGtt-3′ of SEQ ID NO:2. A medicine for inhibiting cancer growth and spread includes a medicinal preparation including miR-520b substrate and medical agent wherein the miR-520b substrate is miR-520b sequence and has concentration of 50 μM, and the miR-520b sequence is 5′-AAAGUGCUUCCUUUUAGAGGGtt-3′ of SEQ ID NO:2.

### BACKGROUND OF THE INVENTION

### 1. Field of the Invention

The invention relates to technologies for inhibiting cancer growth and spread and more particularly to a miR-520b sequence for inhibiting cancer growth and spread and medicine therefor.

### 2. Description of Related Art

Cancer is malignant tumors and is the first of ten leading causes of death in Taiwan. Cancer is a group of diseases involving abnormal cells growth with the potential to invade or spread to other parts of the body. Cancer is often treated with some combination of radiation therapy, surgery, chemotherapy, and targeted therapy. The latest and most effective treatment to most cancer patients involves less surgery and combining radiation therapy and chemotherapy. It is understood that above treatment is not effective to some cancer patients. This is because cancer cells are resistant to chemotherapy and radiation therapy and to the worse, the cancer cells may spread to other parts of the human body. There is no effective treatment at the end stage of cancer. It is found that the cancer survival rate does not increase greatly due to the advancement of medical technologies.

It is desired to find an element for inhibiting cancer growth and spread and increasing efficacy of radiation therapy and chemotherapy as a replacement or in addition to the typical cancer treatment.

### SUMMARY OF THE INVENTION

It is therefore one object of the invention to provide a miR-520b sequence for inhibiting cancer growth and spread wherein the miR-520b sequence is 5′-AAAGUGCUUCCUUUUAGAGGGtt-3′ of SEQ ID NO:2.

It is another object of the invention to provide a medicine for inhibiting cancer growth and spread comprising a medicinal preparation including miR-520b substrate and medical agent wherein the miR-520b substrate is miR-520b sequence and has concentration of 50 μM, and the miR-520b sequence is 5′-AAAGUGCUUCCUUUUAGAGGGtt-3′ of SEQ ID NO:2.

The above and other objects, features and advantages of the invention will become apparent from the following detailed description taken with the accompanying drawings.

### BRIEF DESCRIPTION OF THE DRAWINGS

FIG. 1 is a bar chart showing relative expression (fold) versus SAS (sarcoma amplified sequence) cells and OECM1 (oral epidermal cells-M1) cells for pcDNA (plasmid cytomegalovirus deoxyribonucleic acid) and miR-520b according to the invention, and showing a great increase of relative expression of miR-520b due to the transfected miR-520b;

FIG. 2A is a line chart showing coverage (%) versus time for pcDNA and miR-520b of OECM1 cells of the invention, and showing miR-520b sequence capable of inhibiting cancer cells from spreading;

FIG. 2B is a line chart showing coverage (%) versus time for pcDNA and miR-520b of SAS cells of the invention, and showing miR-520b sequence capable of inhibiting cancer cells from spreading;

FIG. 3A is a bar chart showing relative level (fold) versus pcDNA and miR-520b for pcDNA and miR-520b of OECM1 cells of the invention, and showing miR-520b sequence capable of inhibiting cancer cells from spreading;

FIG. 3B is a bar chart showing relative level (fold) versus pcDNA and miR-520b for pcDNA and miR-520b of SAS cells of the invention, and showing miR-520b sequence capable of inhibiting cancer cells from spreading;

FIG. 4 contains two line charts in which one shows survival fraction (fold) versus cisplatin (μg/ml) for pcDNA and miR-520b of OECM1 cells and the other shows survival fraction (fold) versus cisplatin (μg/ml) for pcDNA and miR-520b of SAS cells of the invention, and shows the transfected miR-520b sequence capable of increasing efficacy of the anti-cancer drug cisplatin in cancer treatment and decreasing survival fraction of cancer cells;

FIG. 5A contains a line chart showing survival fraction (fold) versus Gy for pcDNA and miR-520b of OECM1 cancer cells of the invention and an image of cells presented in the form of Gy versus pcDNA and miR-520b of the invention, and shows the transfected miR-520b sequence capable of increasing efficacy of radiation therapy and decreasing survival fraction of cancer cells;

FIG. 5B contains a line chart showing survival fraction (fold) versus Gy for pcDNA and miR-520b of cancer SAS cancer cells of the invention and an image of cells presented in the form of Gy versus pcDNA and miR-520b of the invention, and shows the transfected miR-520b sequence capable of increasing efficacy of radiation therapy and decreasing survival fraction of cancer cells;

FIG. 6A contains a bar chart showing number of spheres versus control and miR-520b, and two images of controls and two images of miR-520b of OECM1 of the invention, and showing miR-520b sequence capable of inhibiting the growth of head and neck OECM1 cancer cells;

FIG. 6B contains a bar chart showing number of spheres versus control and miR-520b, and two images of controls and two images of miR-520b of SAS of the invention, and showing miR-520b sequence capable of inhibiting the growth of head and neck SAS cancer cells;

FIG. 6C contains a bar chart showing number of spheres versus control and miR-520b, and two images of controls and two images of miR-520b of SW480 of the invention, and showing miR-520b sequence capable of inhibiting the growth of SW480 colon cancer cells;

FIG. 6D contains a bar chart showing number of spheres versus control and miR-520b, and two images of controls and two images of miR-520b of MDA-MB231 of the invention, and showing miR-520b sequence capable of inhibiting the growth of MDA-MB231 breast cancer cells;

FIG. 7A contains a bar chart showing relative expression versus OECM1, SAS, and Fadu for pcDNA and miR-520b, two images of OECM1 in the form of V, 520b versus CD44 GAPDH, two images of SAS in the form of V, 520P versus CD44 GAPDH, and two images of Fadu in the form of V 520b versus CD44 GAPDH of the invention, and shows decrease of relative expression of gene CD44 as target in OECM1, SAS, and Fadu cells of the transfected miR-520b sequence in which V is vector control, i.e., pcDNA; 520b is miR-520b in FIGS. 1-5; and V and 520b are used in this figure due to less space for indications.

FIG. 7B contains a bar chart showing relative expression (firefly/renilla RLU) versus WT UTR for Vector and miR-520b of OECM1 and a bar chart showing relative expression (firefly/renilla RLU) versus WT UTR for Vector and miR-520b of SAS of the invention by using EL (electroluminescent) device, and shows miR-520b sequence capable of disposing in three non-transfection regions of the targeted gene CD44 so as to adjust the relative expression;

FIG. 8A contains a line chart of tumor size (mm³) versus days for miR-520b, control, anti-520b, and P<0.0001, an image of control, an image of anti-520b, and an image of miR-520b as a result of using mice in an experiment of the invention for showing miR-520b sequence capable of inhibiting cancer growth and spread in which Fadu having high relative expression or capable of inhibiting miR-520b is injected into two sides of the back of the mouse to embed 1×10⁵ by hypodermic injection, representative tumor and average tumor size are shown in the images, miR-520b is capable of inhibiting cancer growth, and * represents P<0.0001; and

FIG. 8B contains a chart of number of nodules versus anti-520b, control and miR-520b for metastasis nodules, an image of anti-520b, an image of control, and an image of miR-520b a result of using mice in an experiment of the invention for showing miR-520b sequence capable of inhibiting cancer growth and spread in which Fadu having high relative expression or capable of inhibiting miR-520b is injected into the respiratory system of the mouse to embed 5×10⁵ cells by tail vein injection, representative the image representing the mouse losing liver and the image representing number of nodules per the liver of the mouse are shown, and miR-520b sequence is capable of decreasing the number of cancer cells being spread.

### DETAILED DESCRIPTION OF THE INVENTION

The invention is directed to a miR-520b sequence for inhibiting cancer growth and spread wherein the miR-520b sequence is 5′-AAAGUGCUUCCUUUUAGAGGGtt-3′ of SEQ ID NO:2 and an inversion of the miR-520b sequence is 5′-CCCUCUAAAA GGAAGCACUUU-3′ of SEQ ID NO:1.

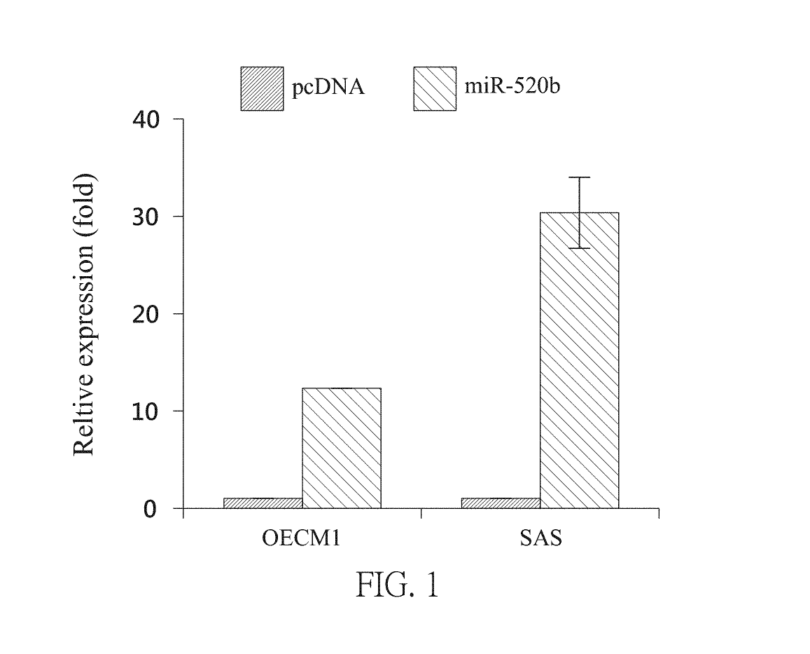

Referring to FIGS. 1 to 8B, a miR-520b sequence for inhibiting cancer growth and spread in accordance with the invention is illustrated below. In detail, miR-520b sequence is transferred to cancer cells so as to increase relative expression in the cells. Thereafter, an observation of the affected cancer cells is made possible. As shown in FIG. 1, miR-520b can greatly increase relative expression of miR-520b. The relative expression in OECM1 cells is 12.4 times greater than the control (pcDNA) and the relative expression in SAS cells is 30.3 times greater than the control (pcDNA).

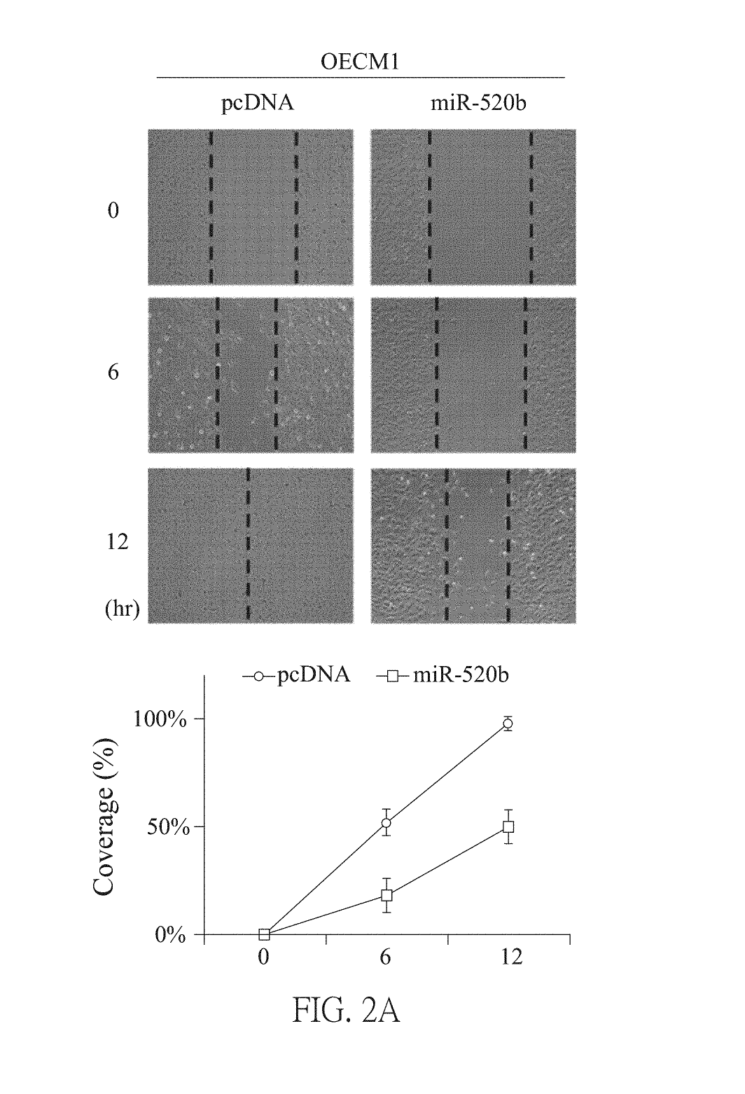

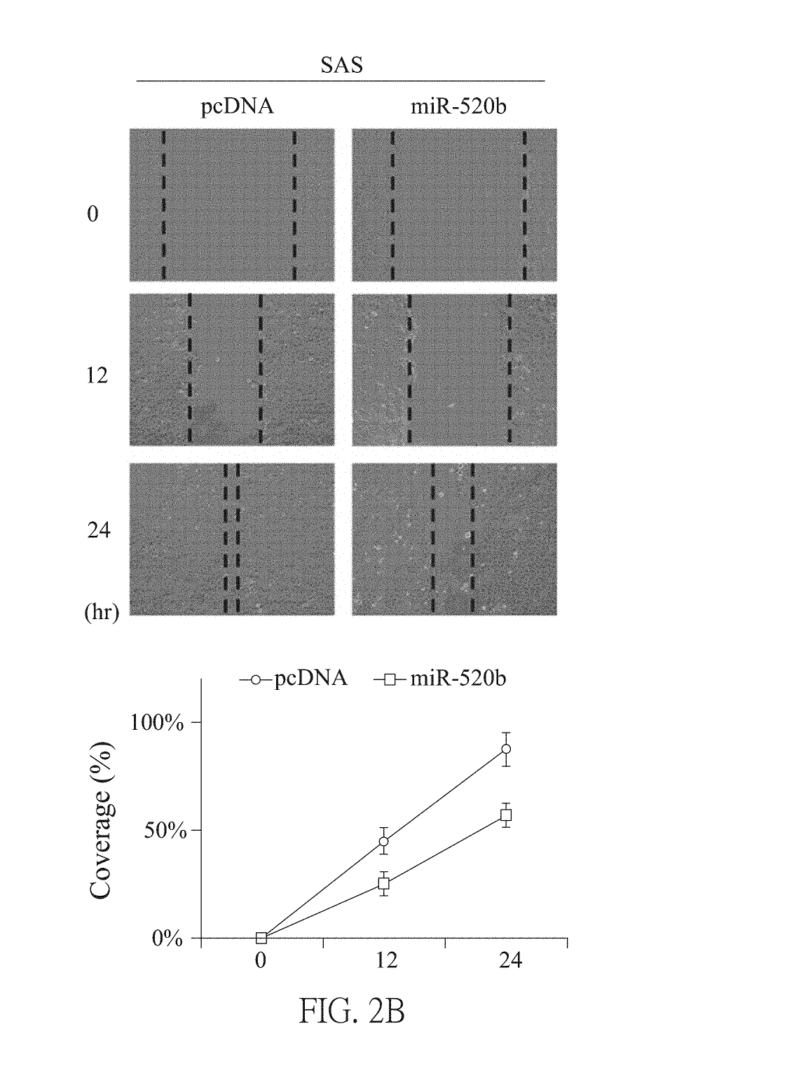

Thereafter, cells motility experiment is conducted for evaluating influence of miR-520b to cancer cells. Results are shown in FIGS. 2A and 2B. After OECM1 and SAS cells are transfected by miR-520b sequence, it is found that speed of moving cells to gaps is decreased greatly. In OECM1 cells, all gaps are occupied by control in 12 hours and 51% of gaps are occupied by miR-520b as transfection. Similarly, in SAS cells, 98% of gaps are occupied by control in 24 hours and 64% of gaps are occupied by miR-520b as transfection.

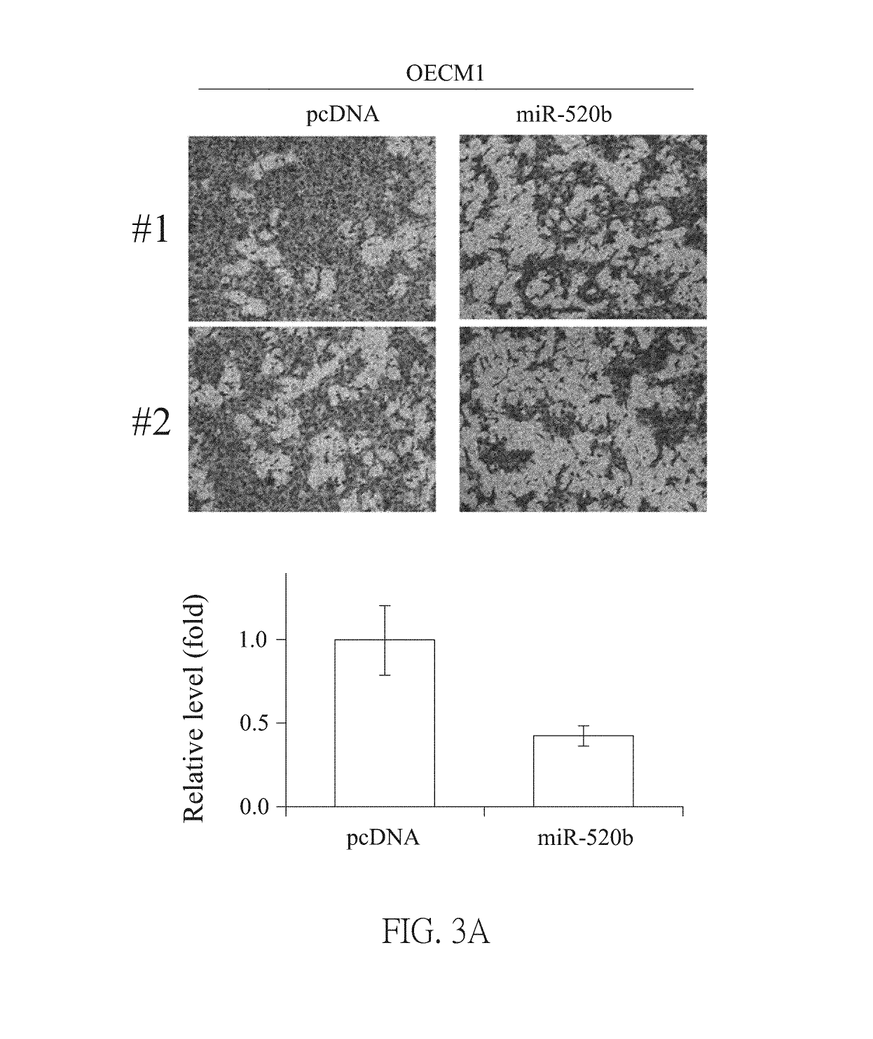

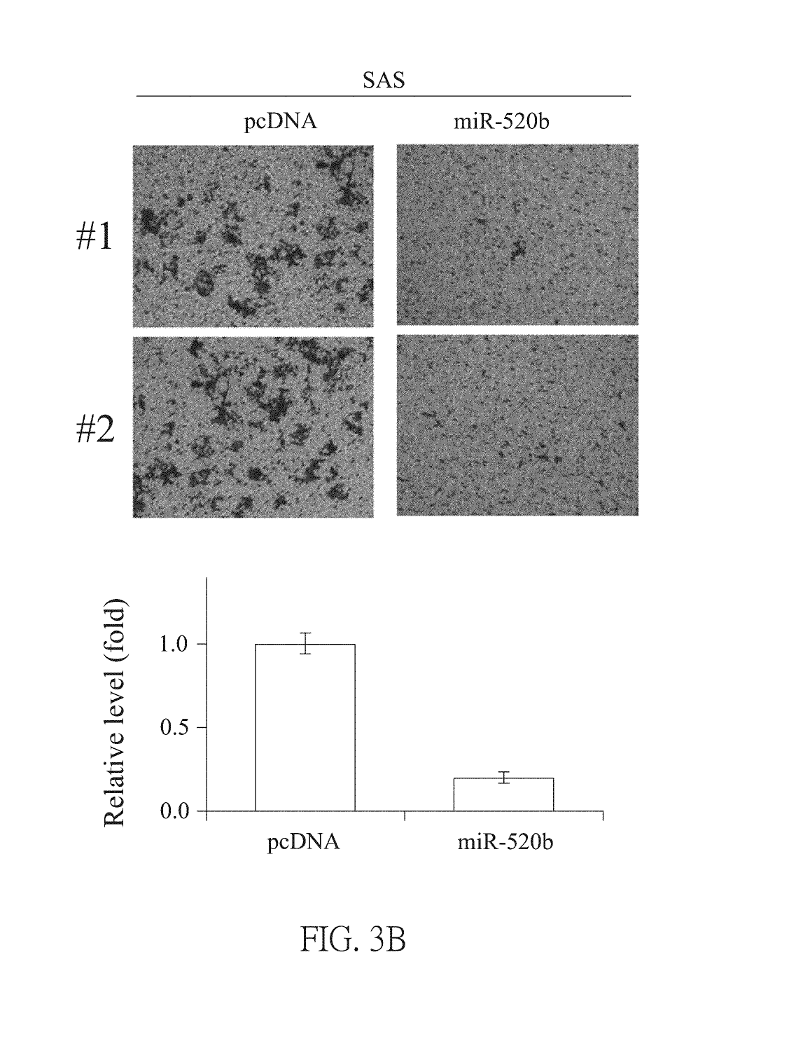

In a substrate membrane invasion experiment, capability of surrounding tissues being attacked by cancer cells is evaluated. Results are shown in FIGS. 3A and 3B. In OECM1 cells which are transfected by miR-520b sequence, the number of cells passes through the substrate membrane to the underlying tissues is decreased by 57%. Similarly, in SAS cells which are transfected by miR-520b sequence, the number of cells passes through the substrate membrane to the underlying tissues is decreased by 80%. It is abundantly clear that cancer cells transfected by miR-520b sequence has a decreased capability of passing through the substrate membrane.

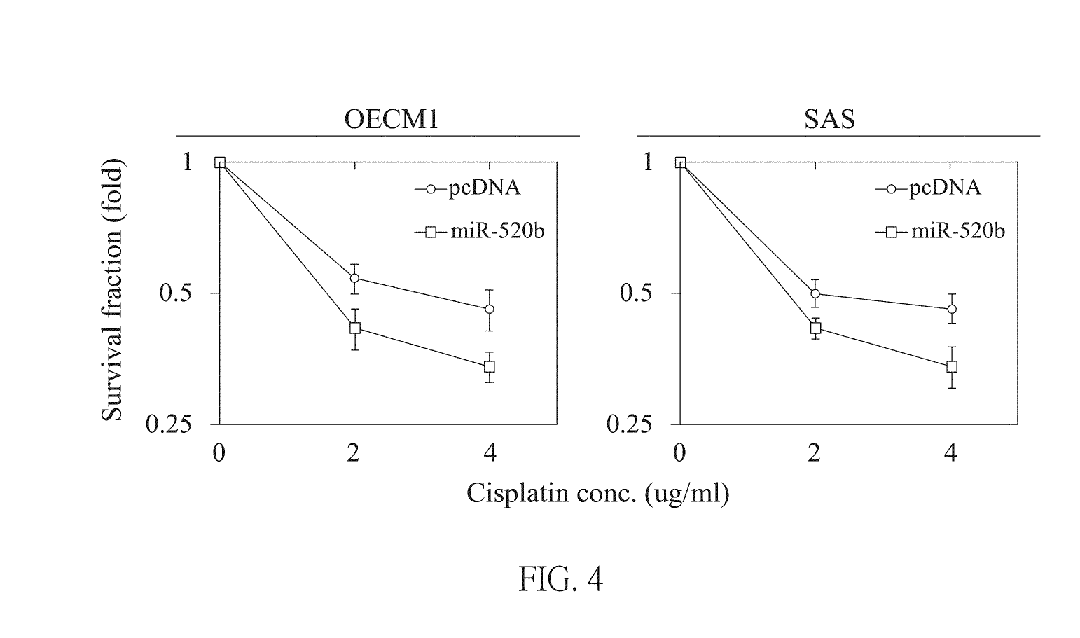

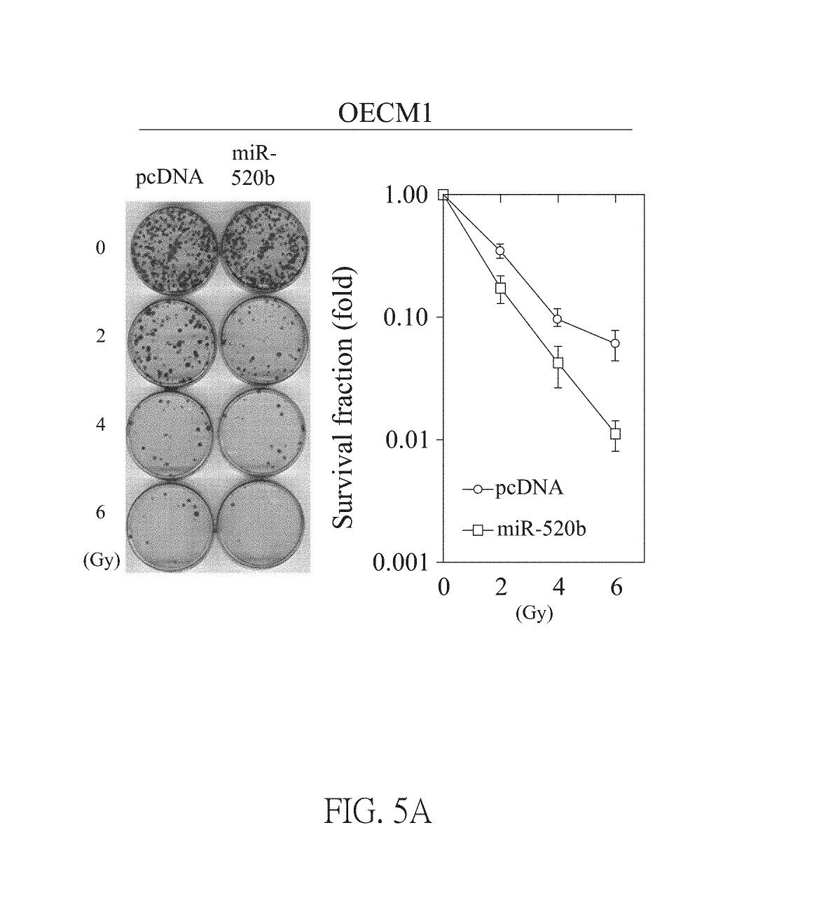

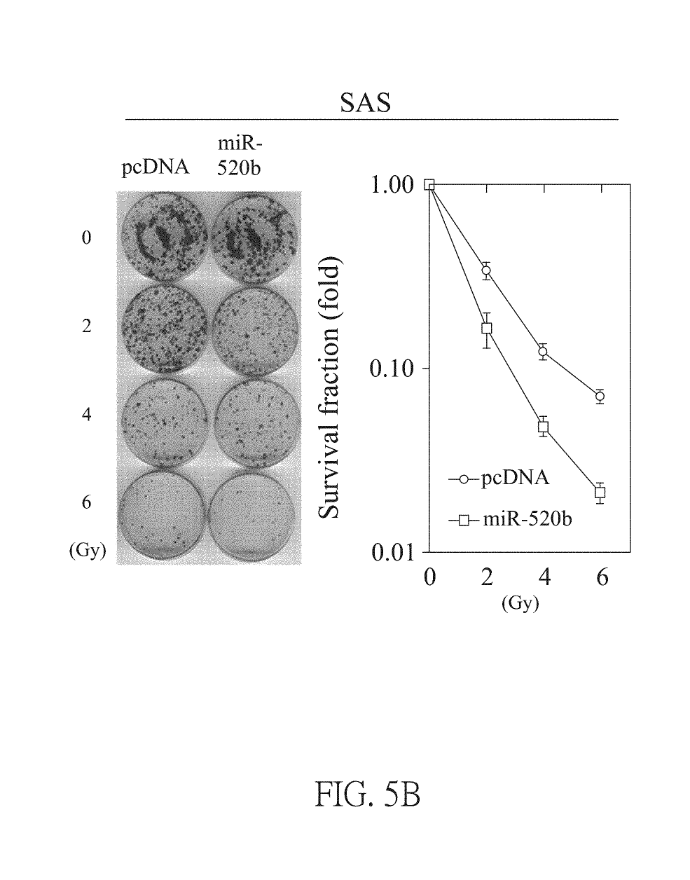

Also, whether cells transfected by miR-520b sequence has resistance to chemotherapy and radiation therapy is evaluated. 0-4 μg/ml cisplatin or 0-6 Gy of radiation is applied to the cells transfected by miR-520b sequence. Next, the number of the survived cells or groups of the survived cells is observed and results are shown in FIG. 4. It is found that the anti-cancer drug cisplatin is very effective in killing cancer cells with the number of the survived cancer cells greatly decreased. For example, for OECM1 cells with application of 0-4 μg/ml dosage of cisplatin, the number of cells being killed is increased 1.26 times and for SAS cells with application of 0-4 μg/ml dosage of cisplatin, the number of cells being killed is increased 1.44 times. Similarly, efficacy of killing cancer cells by radiation therapy is shown in FIGS. 5A and 5B. As shown, after being transfected by miR-520b sequence, the cells exhibit less resistance to radiation therapy, thereby greatly decreasing the group of the survived cells. For OECM1 cells, with 6 Gy of radiation being applied, the number of cells being killed is increased 1.82 times, and for SAS cells, with 6 Gy of radiation being applied, the number of cells being killed is increased 1.7 times. In other words, the miR-520b sequence can decrease resistance of cancer cells to chemotherapy and radiation therapy, thereby decreasing the growth of cancerous tumors.

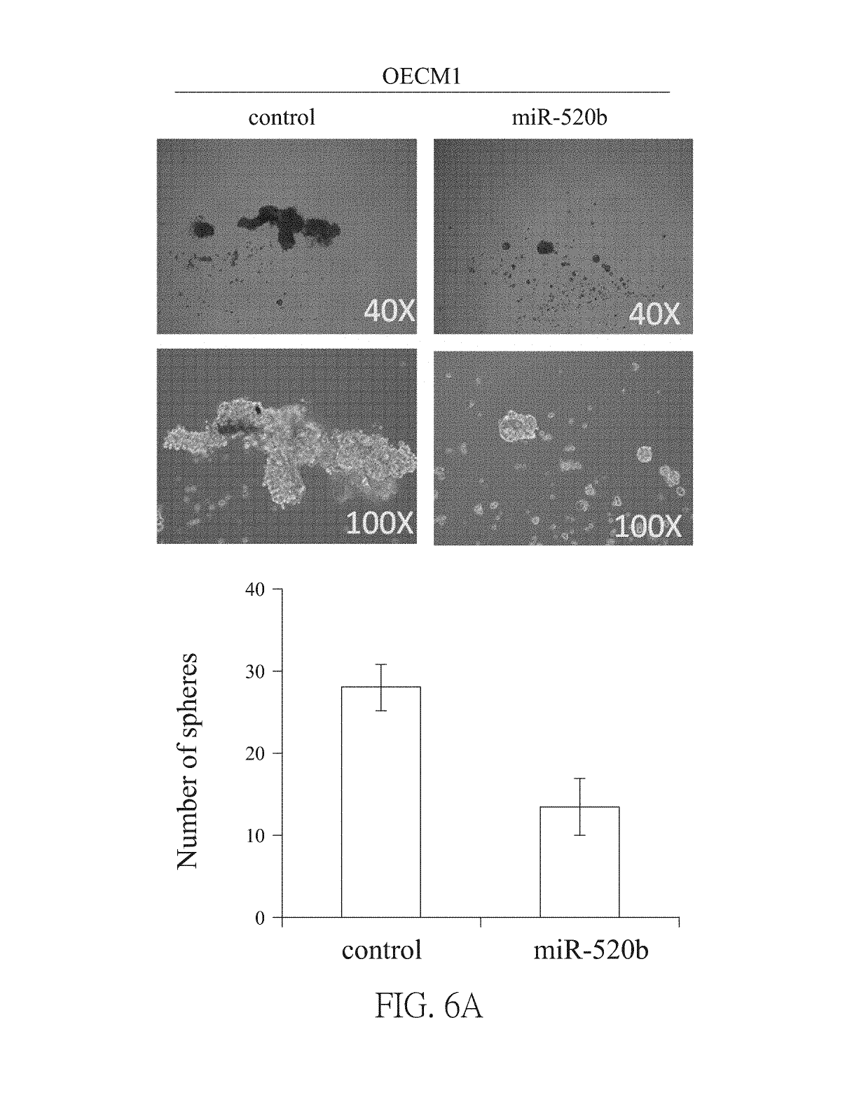

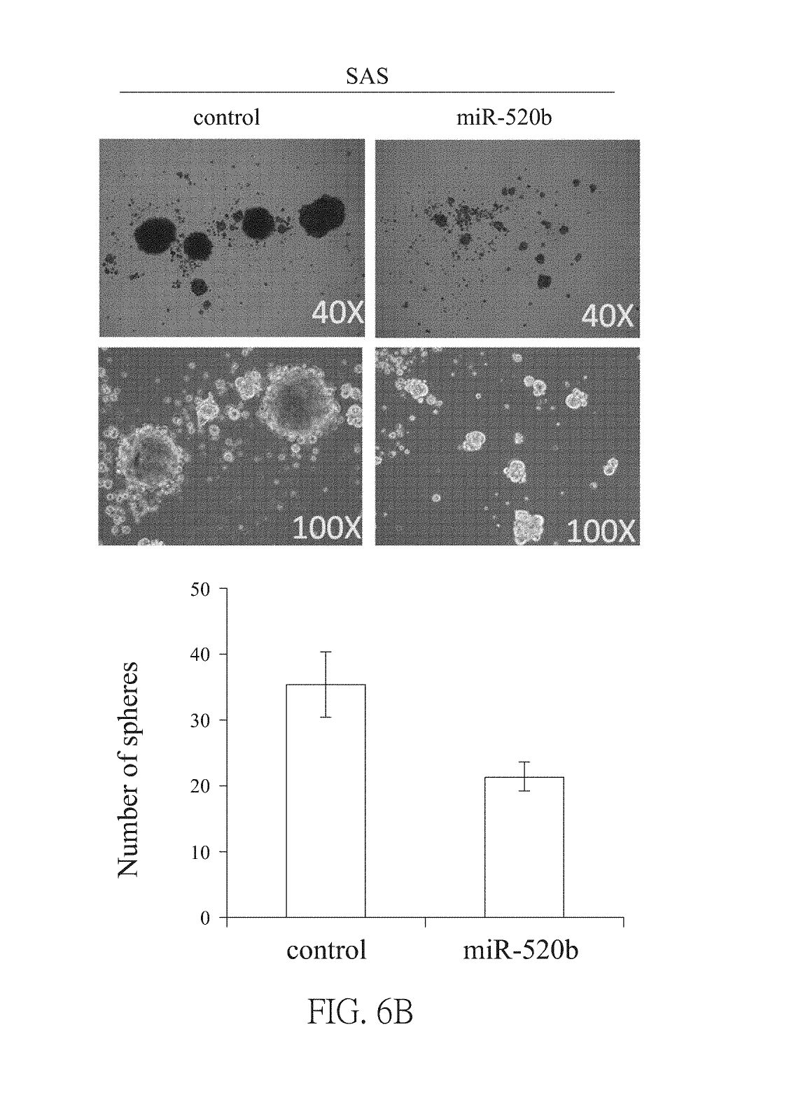

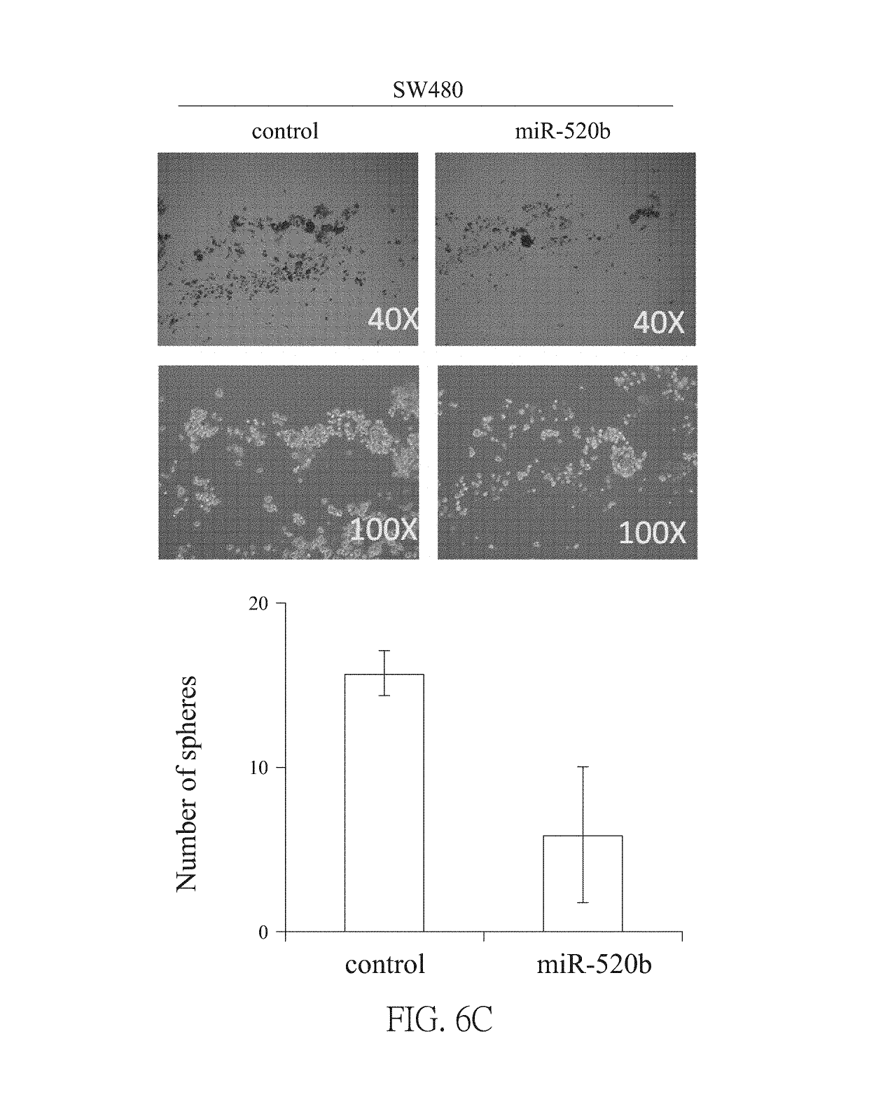

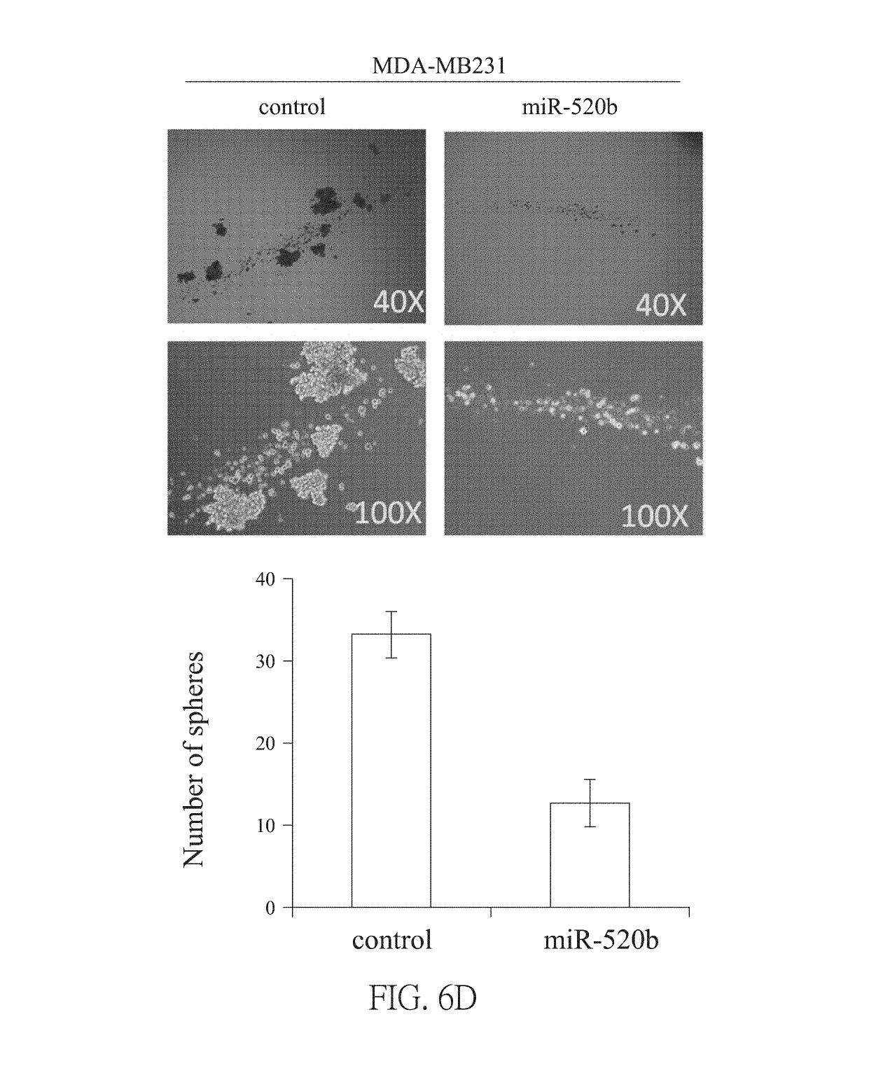

Mode of researching cells is used in cell sphere formation to evaluate in vitro tumorigenesis. Results are shown in FIGS. 6A and 6B. For OECM1 and SAS cells transfected by miR-520b sequence, the number of grown cells is decreased about 50%.

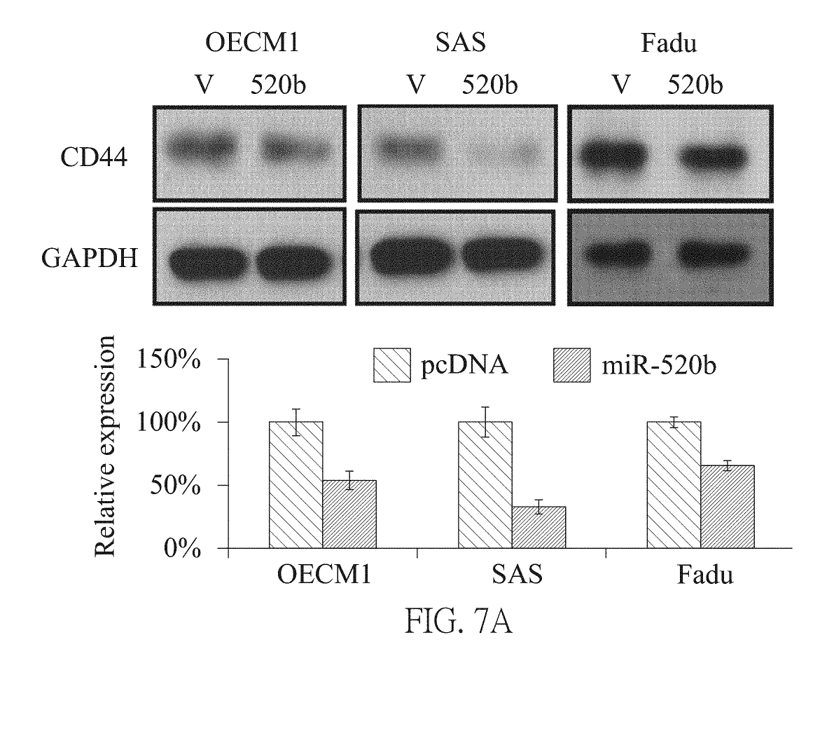

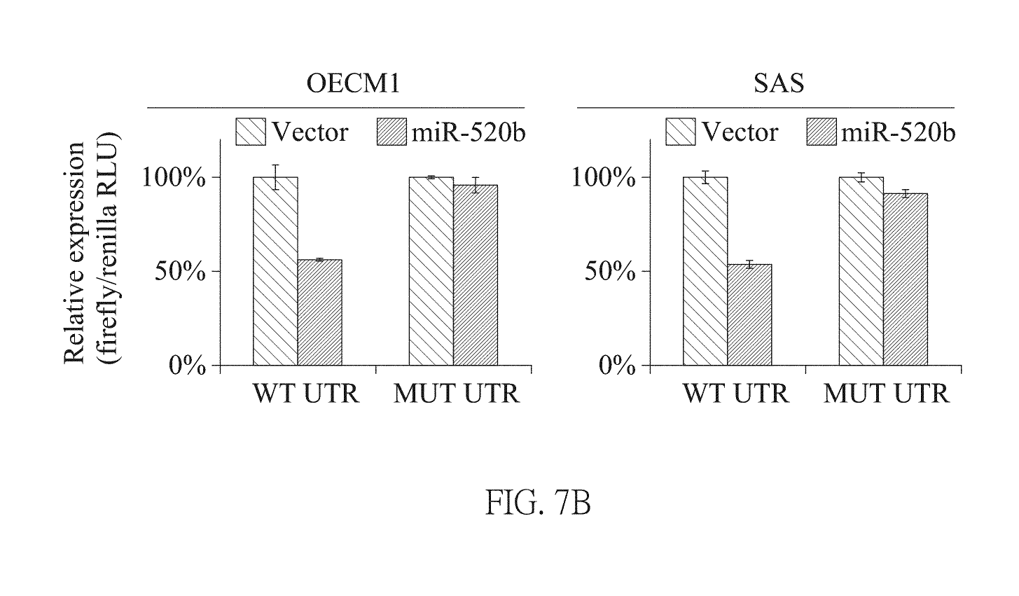

Western blot and EL device are used to prove whether gene CD44 is a targeted gene of miR-520b sequence or not. Relative expression of CD44 is shown in FIG. 7A. The transfected miR-520b can decrease relative expression of CD44. In OECM1, SAS and Fadu cells, the relative expression of CD44 is decreased about 34 to 67% due to the miR-520b sequence. Results of gene experiment conducted by EL device are shown in FIG. 7B. In the gene tested by EL device, there are three non-transfection regions of wild (or mutated) gene CD44 which is further transfected by miR-520b. The mobility of wild gene CD44 is decreased due to generation of EL and the mobility of mutated gene CD44 is not changed. In OECM1 and SAS cells, the decrease percentages are 44% and 47% respectively.

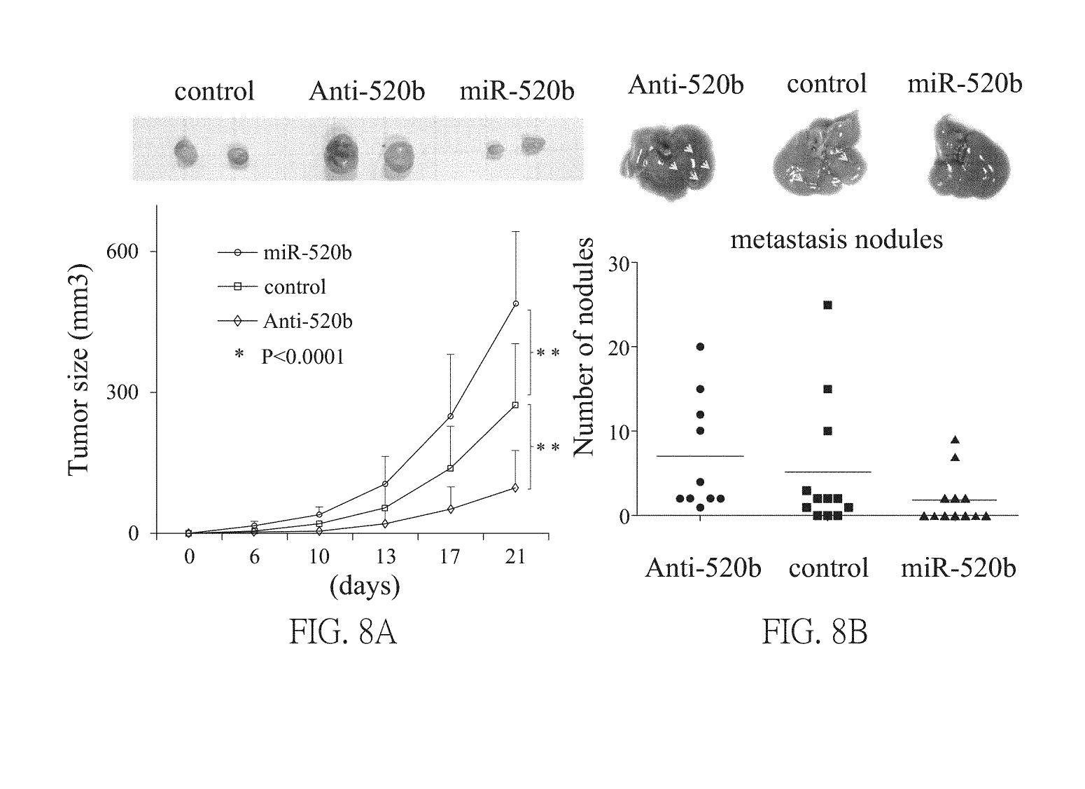

We used mice in an experiment to study whether miR-520b sequence has effect in inhibiting growth and spread of cancer in the body or not, thereby evaluating its potential as a medicine for treating cancer. In the research of tumors growth, cancer cells are transfected by miR-520b sequence of SEQ ID NO:2, an inversion of the miR-520b sequence of SEQ ID NO:1, and control oligonucleotide of SEQ ID NO:3 and SEQ ID NO:4. Next, the cancer cells are injected into skins of the mice. After six days, tumors are grown. The mice are divided into three groups. Cells transfected by miR-520b sequence of SEQ ID NO:2, anti-520b of SEQ ID NO:1, control oligonucleotide of SEQ ID NO:3, and control oligonucleotide of SEQ ID NO:4 are injected into the mice from tail vein two times per week. Observation continues for up to 21 days. Results are shown in FIG. 8A. In comparison with the control, the mice of the experiment group transfected by miR-520b sequence can completely inhibit the growth of tumors under the skin. To the contrary, the mice of the experiment group transfected by the inverse of miR-520b sequence have more grown cancer cells and the tumor size is 1.6 times greater than tumor size of the mice of the control at the end of the 21-day period. With respect to the research of cancer spread, transfected cancer cells are injected into the blood vessel of the mice from tail vein. Six days are waited for simulating the condition of spreading cancer cells to the blood circulation system of the mice. The mice are divided into three groups. Cells transfected by miR-520b sequence of SEQ ID NO:2, Anti-520b of SEQ ID NO:1, control oligonucleotide of SEQ ID NO:3, and control oligonucleotide of SEQ ID NO:4 are injected into the mice from tail vein two times per week. Observation of liver colonization continues for up to 21 days. Results are shown in FIG. 8B. As a comparison, both the control and the mice of the experiment group transfected by the inversion of the miR-520b sequence exhibit liver colonization. As another comparison, the mice of the experiment group can greatly decrease cancer cells' liver colonization. It is concluded that miR-520b sequence can effectively inhibit the growth and spread of cancerous tumors.

In the above description, molecules of the miR-520b sequence having a concentration of 50 μM is dissolved in normal saline. In the experiment of using mice, 50 μl of the saline is injected into the mouse per injection. That is, 20 ng of saline is injected into the mouse (i.e., 1 μg/Kg).

A medicine for inhibiting cancer growth and spread comprising a medicinal preparation is also provided by the invention. The medicinal preparation comprises miR-520b substrate and medical agent. The miR-520b substrate is miR-520b sequence and has concentration of 50 μM. The miR-520b sequence is injected into cancer cells. The miR-520b sequence is 5′-AAAGUGCUUCCUUUUAGAGGGtt-3′ of SEQ ID NO:2. The miR-520b sequence has the functions of inhibiting spread, motility, invasion, and growth of cancer cells, and increasing efficacy of treatment by radiation therapy and chemotherapy.

It is concluded that the miR-520b sequence of the invention has the following advantages: Decreasing cancer cells' resistance to radiation therapy and chemotherapy, thereby killing cancer cells, inhibiting cancer cells' motility and invasion, decreasing the growth of migrating cancer cells, and exhibiting the capability of miR-520b sequence in inhibiting the growth and spread of cancer cells. Further, it is found in the animal experiment that hypodermic cancer tumors are significantly decreased by injecting miR-520b sequence into the skin of the mouse. Also, liver colonization of the cancerous cells is decreased. All of above demonstrates the capability of the miR-520b sequence in treating cancer.

While the invention has been described in terms of preferred embodiments, those skilled in the art will recognize that the invention can be practiced with modifications within the spirit and scope of the appended claims.

1. A method for inhibiting cancer growth and spread, comprising the steps of:
(a) modifying the human miR-520b nucleic acid molecule sequence by adding a tt nucleic acid sequence to the -3′ end of the human miR-520b nucleic acid molecule sequence, thus forming a modified miR-520b nucleic acid molecule sequence 5′-AAAGUGCUUCCUUUUAGAGGGtt-3′ of SEQ ID NO:2; and
(b) exposing cancer cells to said modified miR-520b nucleic acid molecule sequence 5′-AAAGUGCUUCCUUUUAGAGGGtt-3′, wherein said modified miR-520b nucleic acid molecule sequence 5′-AAAGUGCUUCCUUUUAGAGGGtt-3′ affects the cancer cells behavior.

2. The method of claim 1, wherein in said step (b), said modified miR-520b nucleic acid molecule sequence 5′-AAAGUGCUUCCUUUUAGAGGGtt-3′ affects the cancer cells behavior by decreasing the cancer cells' resistance to radiation therapy and chemotherapy, thereby decreasing growth and motility of the cancer cells.

3. The method of claim 1, further comprising the step of:
increasing relative expression of said modified miR-520b nucleic acid molecule sequence 5′-AAAGUGCUUCCUUUUAGAGGGtt-3′ in the cancer cells by means of substrate expression or transfecting said modified miR-520b nucleic acid molecule sequence 5′-AAAGUGCUUCCUUUUAGAGGGtt-3′.

4. A method for inhibiting cancer growth and spread, comprising the steps of:
(a) modifying the human miR-520b nucleic acid molecule sequence by adding a tt nucleic acid sequence to the -3′ end of the human miR-520b nucleic acid molecule sequence, thus forming a modified miR-520b nucleic acid molecule sequence 5′-AAAGUGCUUCCUUUUAGAGGGtt-3′; and
(b) fabricating a medicinal substance for treating cancer, said medicinal substance including a modified miR-520b substrate and a medical agent, wherein said modified miR-520b substrate includes said modified miR-520b nucleic acid molecule sequence 5′-AAAGUGCUUCCUUUUAGAGGGtt-3′ having concentration of 50 μM.

5. The method of claim 4, further comprising the steps of:
dissolving the molecules of the modified miR-520b sequence 5′-AAAGUGCUUCCUUUUAGAGGGtt-3′ in normal saline, thus forming a medicinal solution, and
injecting said medicinal solution into the cancer cells in proportion of 1 μg of said medicinal solution per 1 kG of a patient weight.

6. The method of claim 4, further comprising the step of:
inhibiting the cancer cells growth as the result of injecting said medicinal solution into said cancer cells.

7. The method of claim 4, further comprising the step of:
inhibiting cancer cells motility and invasion as the result of injecting said medicinal solution into said cancer cells.

8. The method of claim 4, further comprising the step of:
treating head and neck cancer cells by injecting said medicinal solution into the cancer cells.

9. The method of claim 4, further comprising the step of:
increasing efficacy of radiation therapy as the result of injecting said medicinal solution into the cancer cells.

10. The method of claim 4, further comprising the steps of:
increasing efficacy of chemotherapy as the result of injecting cancer cells with said medicinal solution.

11. A medicinal substance for treating cancerous cells, comprising:
modified miR-520B nucleic acid molecules, each having a modified sequence 5′-AAAGUGCUUCCUUUUAGAGGGtt-3′ containing a tt nucleic acid sequence added at the -3′ end of the human miR-520b nucleic acid molecule sequence,
wherein, upon application to cancerous cells, said modified miR-520b nucleic acid molecules having said modified sequence 5′-AAAGUGCUUCCUUUUAGAGGGtt-3′, affect the cancerous cells behavior through a mechanism selected from a group including: inhibiting cancerous cells growth and spread, inhibiting cancerous cells motility and invasion, decreasing resistance to radiation therapy, decreasing resistance to chemotherapy, and combinations thereof.

12. The medicinal substance of claim 11, further including a modified miR-520b substrate and a medical agent, wherein said modified miR-520b substrate includes said modified miR-520b nucleic acid molecule sequence 5′-AAAGUGCUUCCUUUUAGAGGGtt-3′ having a concentration of 50 μM.

13. The medicinal substance of claim 11, further including a medicinal solution formed by dissolving the molecules having said modified miR-520b sequence 5′-AAAGUGCUUCCUUUUAGAGGGtt-3′ in normal saline,
said medicinal solution for injection into the cancerous cells in proportion of 1 μg of said medicinal solution per 1 kG of a patient weight.


SEQUENCE LISTING (4 sequences)

SEQ ID NO: 1 - RNA, 21 nt, Artificial
  single strand RNA, complement to Hsa-miR-520b
  primer_bind (1)..(21)
cccucuaaaaggaagcacuuu

SEQ ID NO: 2 - DNA, 23 bp, Artificial
  double strand oligonucleotide, same sequence to hsa-miR-520b add TT base at 3′ end
  snRNA (1)..(23)
aaagugcuuccuuuuagagggtt

SEQ ID NO: 3 - RNA, 21 nt, Artificial
  single strand RNA, Negative control for SEQUENCE NO 1
  primer_bind (1)..(21)
caguacuuuuguguaguacaa

SEQ ID NO: 4 - DNA, 21 bp, Artificial
  double strand oligonucleotide, Negative control for SEQUENCE NO 2
  snRNA (1)..(21)
uucuccgaacgugucaugutt